(19) United States
(12) Patent Application Publication
(10) Pub. No.: US 2007/0203520 A1
(43) Pub. Date: Aug. 30, 2007

(54) Endovascular filter

(76) Inventors: Dennis Griffin, Englewood, CO (US); Arne Molgaard-Nielsen, Oesterbro (DK); Anthony O. Ragheb, West Lafayette, IN (US); Raymond B. Leonard II, Bloomington, IN (US); Neal E. Fearnot, West Lafayette, IN (US); William D. Voorhees III, West Lafayette, IN (US); Thomas G. Kozma, McHenry, IL (US); Brian L. Bates, Bloomington, IN (US); Thomas A. Osborne, Bloomington, IN (US)

Correspondence Address: BRINKS HOFER GILSON & LIONE/CHICAGO/COOK, PO BOX 10395, CHICAGO, IL, 60610, US

(21) Appl. No.: 11/654,278

(22) Filed: Jan. 16, 2007

Related U.S. Application Data
(63) Continuation-in-part of application No. 10/172,725, filed on Jun. 14, 2002.
(63) Continuation-in-part of application No. 10/223,415, filed on Aug. 19, 2002.
Application No. 10/223,415 is a continuation-in-part of application No. 09/027,054, filed on Feb. 20, 1998, now Pat. No. 6,774,278.
Application No. 10/223,415 is a continuation-in-part of application No. 08/645,646, filed on May 16, 1996, now Pat. No. 6,096,070.
Application No. 08/645,646 is a continuation-in-part of application No. 08/484,532, filed on Jun. 7, 1995, now Pat. No. 5,609,629.
(60) Provisional application No. 60/298,803, filed on Jun. 14, 2001.
(60) Provisional application No. 60/038,459, filed on Feb. 20, 1997.

(51) Int. Cl. A61M 29/00

(52) U.S. Cl. 606/200

(57) ABSTRACT

Endovascular filter (10) including a plurality of struts (14) with distal ends (18) adapted to anchor the filter to the vessel wall after deployment, such as by having barbs (20), the filter being adapted to be retrieved if desired. Strut distal ends (18) are coated with an antiproliferative agent (40) that inhibits the ingrowth of tissue around the filter, thereby permitting the filter to be retrieved and removed atraumatically after a prolonged period of time, thus extending the useful life of the retrievable filter. Optionally, the proximal end (22) of the filter may also be so coated, or the entire filter.

### CROSS-REFERENCE TO RELATED APPLICATIONS

This application is a continuation-in-part application of: (1) co-pending U.S. patent application Ser. No. 10/172,725, filed Jun. 14, 2002, which claims priority to provisional application Ser. No. 60/298,803; and (2) co-pending U.S. patent application Ser. No. 10/223,415, filed Aug. 19, 2002, which is a continuation-in-part of application of co-pending non-provisional application Ser. No. 09/027,054, filed Feb. 20, 1998, which claimed priority to provisional application Ser. No. 60/038,459, filed Feb. 20, 1997, and which was also a continuation-in-part application of and claimed priority to application Ser. No. 08/645,646, filed May 16, 1996, now U.S. Pat. No. 6,096,070, issued Aug. 1, 2000, which was in turn a continuation-in-part application of and claimed priority to application Ser. No. 08/484,532, filed Jun. 7, 1995, now U.S. Pat. No. 5,609,629, issued Mar. 11, 1997. All of the above-referenced patent applications are incorporated by reference herein in their entirety.

### TECHNICAL FIELD

The present invention relates to medical devices and more particularly to endovascular filters.

### BACKGROUND OF THE INVENTION

In a trauma patient, orthopedic surgery patient, or neuro patient, where the patient is bedridden and not moving, clot frequently forms in the leg veins. Such clot becomes a serious risk of pulmonary embolism if it breaks loose. Recognition of this occurrence has led to the development of vena cava filters which provide protection from migrating clot. While many such filters are permanently deployed in the patient, temporary filters are known that are to be removed when it is determined that the patient is free of the risk of pulmonary embolism. Additionally, retrievable filters are known which may optionally be removed from the patient, if it is determined that the patient is free of the risk of pulmonary embolism within a short period of time after deployment. After deployment of a filter in the patient, proliferating intimal cells begin to grow around the filter struts; after a length of time, such ingrowth prevents removal of the filter without risk of trauma whereafter the filter must remain in the patient. Normally, removal of a filter is only advisable within a couple of weeks after implantation due to intimal proliferation that irreversibly anchors the filter to the vessel wall. See, for example, SCVIR March 2001, San Antonio, Tex., USA, Scientific Session 25 Abstract No. 194, Gimeno, M. S., et al.

In U.S. Pat. No. 5,133,733, a collapsible filter is disclosed that is implantable in a blood vessel of a patient, and in particular in the inferior vena cava. Such filters are utilized during endovascular procedures to entrap thrombi or emboli in the blood that flows through a vein and prevent them from reaching the lungs of a patient and thereby cause pulmonary embolization. Such filters are particularly, but not exclusively, concerned with the inferior vena cava, and have legs or similar structures that anchor to the vessel wall at the desired placement site. Other filters are disclosed in U.S. Pat. Nos. 3,540,431; 3,952,747; 4,425,908 and 4,619,246.

In the first-mentioned patent, a collapsible filter is provided that has limited axial length for facilitating the insertion procedure, with a moderate reduction of the blood flow area of the vein, and in its collapsed state the filter is concentrated into a slender and very narrow bundle of filter elements allowing for a correspondingly slender and narrow insertion catheter. In the expanded condition, four legs extend from an apical hub whereat they are joined together by a ferrule, and each leg of the filter includes a central element, bent into a smooth quasi-halfsinusoidal form, and two substantially symmetrical curved side elements extending on either side of the central element are joined to the hub and to an eyelet surrounding the central element along its length that is slidable along the central element.

The filter of U.S. Pat. No. 5,133,733 as a whole may be folded to a collapsed condition having an outer diameter only about as large as the thicknesses of the metal central and side elements, and then is unfolded from a collapsed insertion condition in which the central elements and side elements of all legs forms a narrow bundle for arrangement in a catheter-like insertion instrument, into a tulip-like filter configuration with the side elements interposed between the central elements of the legs to assume the shape of an apertured solid of evolution with one pointed end at the apical hub. At the free end of each leg central element is a reversely turned anchoring hook engageable with the vessel wall for anchoring the filter in place. In the unfolded tulip-like configuration, the distal ends of the filter legs, both the central and side elements, will engage the wall of the vein along a certain length, minimizing the risk of perforation of the wall, and is said to provide an optimum possibility for filter ingrowth in the vein wall and thereby an optimum long term security against migration of the filter. If the filter needs to be removed after more than fourteen days, the filter ingrowth is an undesirable effect.

It is therefore desired to provide a vena cava filter that is adapted to be removable from its deployed location in a vessel of a patient without trauma to the tissue of the vessel wall and without risk of tearing of intimal tissue which could cause embolization.

It is further desired to provide such a retrievable filter that is adapted for extended retrieval time in a patient, again without risk of trauma.

### SUMMARY OF THE INVENTION

The foregoing problem is solved and a technical advance is achieved in an illustrative endovascular filter for retrievable deployment in a blood vessel of a patient. A plurality of struts extend and diverge from an apical hub at a proximal end to respective distal ends adapted to anchor to the vessel wall when expanded and deployment at a treatment site in a blood vessel of a patient, and lengths of the distal ends of the struts are engageable with and against the vessel wall when deployed. The distal end lengths, and preferably the anchoring sections also, are coated with an antiproliferative agent or bioactive material that prevents or minimizes tissue growth. One such particularly useful bioactive material is paclitaxel, a drug known to have cytostatic properties and that has been shown to inhibit vascular smooth cell migration and proliferation contributing to neointimal hypoplasia.

In an additional aspect, it is preferable to also coat the proximal end of the filter with the antiproliferative agent. Ingrowth would be inhibited were the proximal end to enter into engagement with the vessel wall when the filter becomes misaligned. Likewise, other surface portions of the hub body and side members between the distal and proximal filter ends are preferably coated, were these portions to engage the vessel wall upon misalignment, since the vessel wall may locally protrude inwardly from a linear configuration relative to the filter.

### BRIEF DESCRIPTION OF THE DRAWINGS

An embodiment of the present invention will now be described by way of example with reference to the accompanying drawings, in which:

FIG. 1 discloses an elevation view of an endovascular filter of the present invention in a fully expanded condition;

FIG. 2 is an end view of the expanded filter;

FIG. 3 is an enlargement of one wall-engaging strut distal end that has been treated with an antiproliferative agent;

FIG. 4 is a cross-sectional view through a coated strut end;

FIG. 5 is a view of the filter of FIG. 1 upon deployment in the vena cava;

FIG. 6 illustrates the filter of FIG. 1 being deployed from its delivery system, in the arrangement suitable for a jugular vein approach to the treatment site;

FIG. 7A is an end view of the expanded filter shown in FIG. 1 coated with a bioactive material;

FIG. 7B is a cross section of the portion of a first coating configuration on the coated endovascular filter of FIG. 7A; and

FIG. 7C is a cross-section of a second coating configuration of the coated endovascular filter shown in FIG. 7A.

### DETAILED DESCRIPTION

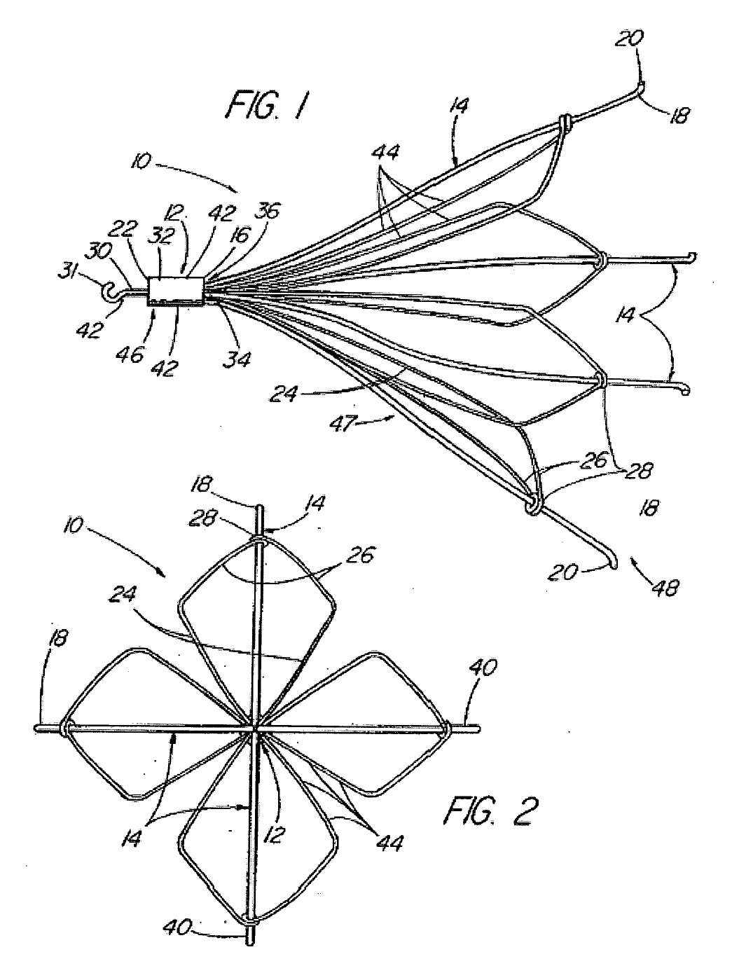

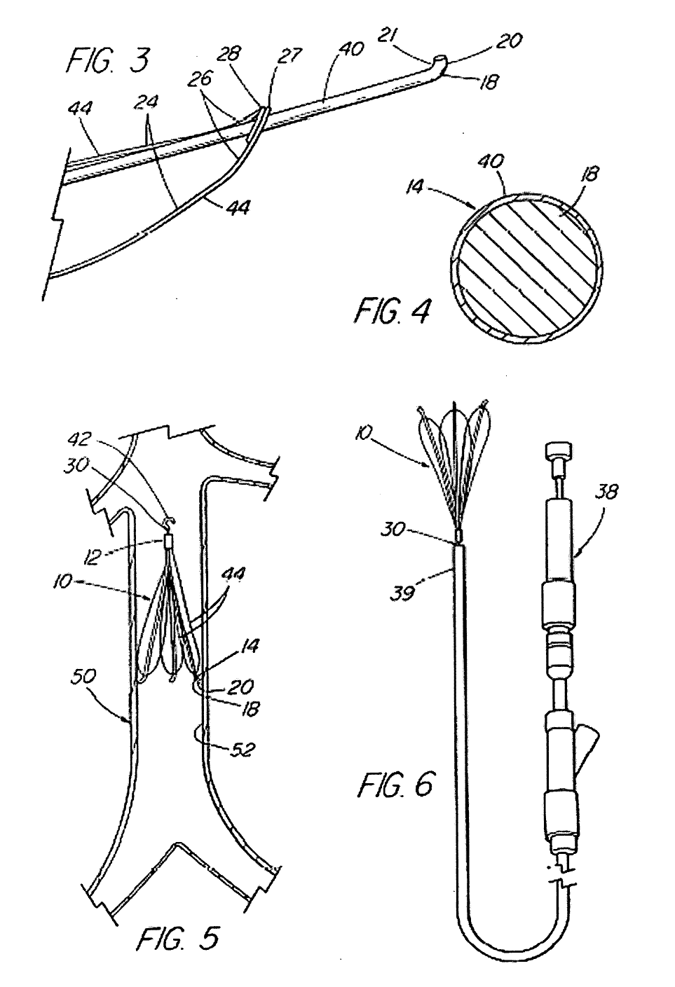

Vena cava filter 10 is shown in FIGS. 1 to 3 in its fully expanded condition to have a proximal portion 46, a medial portion 47 and a distal portion 48. An apical hub body 12, in the proximal portion 46 of the filter 10, has a first or distal end 16 and a second or proximal end 22. A plurality of struts 14 have proximal ends 34 that are secured to the distal end 16 of hub body 12 and have distal end portions 18 that have anchoring sections 20. The struts 14 divergingly extend distally from the distal end 16 of hub body 12. The second or proximal end 22 of hub body 12 has a retrieval section 30 extending therefrom that terminates in a hook 31. The specific embodiment of the filter 10 that is illustrated is shown to have pairs of side elements 24 having proximal ends 36 that are connected to the first end 16 of the hub body 12, each pair of which is associated with a strut 14. The side elements 24 also extend distally in diverging pairs from first end 16 of the hub body 12 and includes distal end portions 26 that converge at 28 and are slidably connected to their associated strut 14. (see FIG. 3) The connection of side elements 24 to the struts 14 preferably being an eyelet 27 that surrounds the strut 14 and is slidable along the strut 14.

Anchoring sections 20 preferably are formed as short hooks 21 that are adapted to press slightly into the wall 52 of a vessel 50 (see FIG. 5) at the deployment site to prevent movement in the direction of blood flow. Apical hub body 12 is adapted to be engaged and retrieved by a retrieval device such as a snare, which can be remotely manipulated to snatch the hook 31 of the retrieval section 30. The retrieval section 30 extends from the second or proximal end 22 of the hub body 12. A ferrule 32 secures the proximal ends 34 of struts 14 and proximal ends 36 of side elements 24, to the hub body 12.

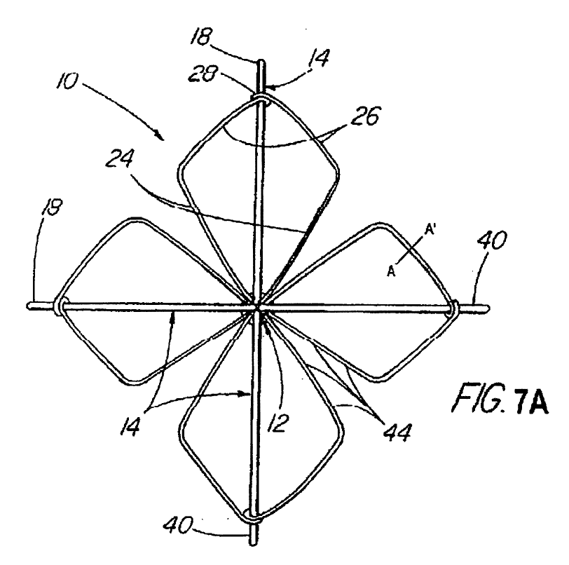

FIG. 6 illustrates the filter 10 being deployed from the catheter 39 of delivery and deployment system 38; the filter has an outermost dimension when in a collapsed state essentially no greater than the combined thicknesses of the hub body, proximal ends 34, 36 of struts 14 and side elements 24, and ferrule 32 therearound, to facilitate assembly into the delivery and deployment system 38 and deployment therefrom. The filter 10 must also be capable of collapsing back to this size so that it can be “swallowed” by a sheath of a retrieval device after the retrieval device snares the hook 31 of the retrieval section 30 during removal from the patient. FIG. 6 shows the arrangement suitable for a jugular vein approach to the treatment site. For a femoral approach, the filter would be reversed in orientation, with the retrieval section 30 being the forwardmost section during delivery. A quite similar filter structure is disclosed in U.S. Pat. No. 5,133,733 and a similar product is sold by William Cook Europe ApS, Bjaeverskov, Denmark as the GÜNTHER TULIP™ Filter, which is designed to be retrievable. Delivery of a filter such as that disclosed in U.S. Pat. No. 5,133,733 is described in detail in U.S. Pat. No. 5,324,304.

At some point after implantation, many patients may resume their mobility and no longer need protection from migrating clot. The current maximum retrieval time after implantation for the GÜNTHER TULIP filter is fourteen days; thereafter, the filter grows into the caval wall, or more precisely, strands of organized thrombus grow around the struts and anchoring sections.

In accordance with the present invention, the distal end sections 18 of struts 14 as well as their anchoring sections 20, are coated with a coating 40 including a bioactive material, such as an antiproliferative or antiinflammatory agent, shown in FIG. 4. Coating 40 may be configured to inhibit or prevent the ingrowth of tissue to and around the distal end portions 18 and anchoring sections 20, at least for an extended length of time after placement, such as for four weeks or more, thereby substantially extending the maximum retrieval time for the filter. This inhibition of ingrowth extends the protection period for the immobile patient, and yet still preserves the eventual retrievability of the filter.

Occasionally an emplaced filter will become misaligned within the vessel, to the extent that the second or proximal end 22 of the hub body 12 will become engaged with the vessel wall 52. While retrieval is still possible although it is more complicated to establish engagement by the retrieval device with the hook 31 of retrieval section 30, it is also desirable to provide a coating of the antiproliferative or antiinflammatory agent 40 to those portions of the filter that may enter into contact with the vessel wall such as portions 42 of the second or proximal end 22 of the hub body 12 including the retrieval section 30 (FIG. 1). Similarly, it may be desirable to provide a coating of agent 40 onto surface portions in the medial portion 44 of the filter including portions of the side elements 24 and struts 14 that are spaced from the distal 48 and proximal 46 filter ends, since the vessel wall 52 may locally “protrude” inwardly because it may not remain truly coaxial around the filter.

One such agent is dexamethasone and related compounds. Another is paclitaxel. Coating of an implantable medical device such as a stent, with a bioactive material, such as paclitaxel, is disclosed in U.S. Pat. No. 6,299,604. It has become well-established that paclitaxel in particular has cytotoxic properties when provided in proper dosages and concentrations, as described in U.S. Pat. No. 6,299,604, and in lower dosages and concentrations would be considered at least cytostatic and therefore able to inhibit neointimal growth, and hence very useful in preventing or inhibiting restenosis.

The coating 40 may be configured to retain a bioactive material, such as a bioactive material that prevents or reduces thrombus formation. Medical devices having an antithrombogenic bioactive material are particularly preferred for implantation in areas of the body that contact blood. An antithrombogenic bioactive material is any bioactive material that inhibits or prevents thrombus formation within a body vessel. The medical device can include any suitable antithrombogenic bioactive material. Types of antithrombotic bioactive materials include anticoagulants, antiplatelets, and fibrinolytics. Anticoagulants are bioactive materials which act on any of the factors, cofactors, activated factors, or activated cofactors in the biochemical cascade and inhibit the synthesis of fibrin. Antiplatelet bioactive materials inhibit the adhesion, activation, and aggregation of platelets, which are key components of thrombi and play an important role in thrombosis. Fibrinolytic bioactive materials enhance the fibrinolytic cascade or otherwise aid is dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Further examples of antithrombotic bioactive materials include anticoagulants such as heparin, low molecular weight heparin, covalent heparin, synthetic heparin salts, coumadin, bivalirudin (hirulog), hirudin, argatroban, ximelagatran, dabigatran, dabigatran etexilate, D-phenalanyl-L-poly-L-arginyl, chloromethy ketone, dalteparin, enoxaparin, nadroparin, danaparoid, vapiprost, dextran, dipyridamole, omega-3 fatty acids, vitronectin receptor antagonists, DX-9065a, CI-1083, JTV-803, razaxaban, BAY 59-7939, and LY-51,7717; antiplatelets such as eftibatide, tirofiban, orbofiban, lotrafiban, abciximab, aspirin, ticlopidine, clopidogrel, cilostazol, dipyradimole, nitric oxide sources such as sodium nitroprussiate, nitroglycerin, S-nitroso and N-nitroso compounds; fibrinolytics such as alfimeprase, alteplase, anistreplase, reteplase, lanoteplase, monteplase, tenecteplase, urokinase, streptokinase, or phospholipid encapsulated microbubbles; and other bioactive materials such as endothelial progenitor cells or endothelial cells.

The coating 40 may be applied by numerous methods, including but not limited to, spraying, dipping, soaking, painting with a brush or similar tool. In the present embodiment the method of coating was spraying as a fine mist. For simplification of fabrication, the entire filter may be so coated.

An excipient (e.g., matrix, binder, carrier, polymer, membrane) may be associated with the active agent and may be under the bioactive layer, over the bioactive layer, mixed with the bioactive layer, or any combination thereof. The excipient material may include, but is not limited to parylene, a cellulose based polymer or a naturally occurring basement membrane material such as Small Intestine Submucosa (SIS).

In the present embodiment, because paclitaxel has low water solubility, no excipient need be used, and the coating may be entirely paclitaxel. The coated device should be handled as gently as possible with minimum scraping, abrading, rubbing, soaking or other physical challenge.

The bioactive material may also be an inhibitor of the molecular target of rapamycin (mTOR), including rapamycin or a rapamycin derivative. The mTOR is believed to be a member of the phosphoinositide 3-kinase related kinase (PIKK) family and a central modulator of cell growth. The mTOR plays a critical role in transducing proliferative signals mediated through the phosphatidylinositol 3 kinase (Pl3K)/protein kinase B (Akt) signaling pathway, principally by activating downstream protein kinases that are required for both ribosomal biosynthesis and translation of key mRNAs of proteins required for G(1) to S phase traverse. One preferred bioactive material is rapamycin (i.e., sirolimus) or a rapamycin derivative (such as tacrolimus, zotarolimus). By targeting mTOR, the immunsuppressant and antiproliferative agent rapamycin inhibits signals required for cell cycle progression, cell growth, and proliferation. Tacrolimus is a macrolide antibiotic. It reduces peptidyl-prolyl isomerase activity by binding to the immunophilin FKBP-12 (FK506 binding protein) creating a new complex. This FKBP12-FK506 complex interacts with and inhibits calcineurin thus inhibiting both T-lymphocyte signal transduction and IL-2 transcription. Tacrolimus and rapamycin are immunosuppressants and immunophilin ligands that inhibit T cell activation. Zotarolimus [40-epi-(1-tetrazolyl)-rapamycin], also called ABT-578, is a semi-synthetic macrolide triene antibiotic derived from rapamycin. Zotarolimus is a potent inhibitor of T-cell lymphocyte proliferation, similar to its precursor rapamycin. Zotarolimus has found exceptional applications in coating cardiovascular stents, especially

A wide range of other bioactive materials can be delivered by the filter, as set forth in U.S. Pat. No. 6,096,070. Accordingly, it is preferred that the bioactive material includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic materials; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic material; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C—, ³H—, ¹³¹I—, ^{32P— or} ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar®, Hytrin® or other agents for treating benign prostatic hyperplasia (BHP) or a mixture of any of these; and various forms of small intestine submucosa (SIS).

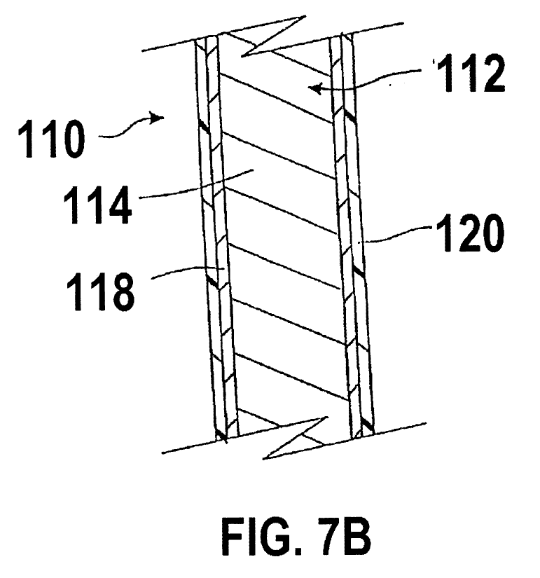

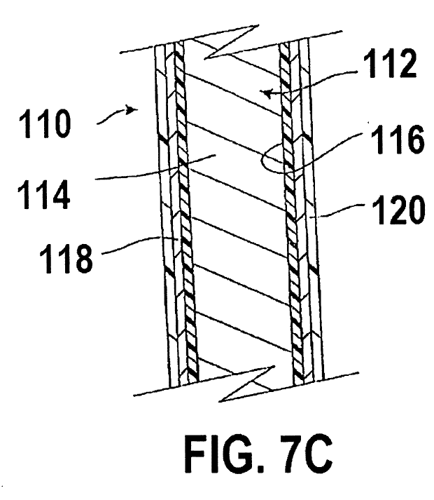

The coating may also be configured to release a bioactive material within a body vessel. FIG. 7A shows an end view of a coated implantable filter. FIG. 7B shows a detailed cross sectional view of a first coating configuration shown along the line A-A′ shown in FIG. 7A. Referring to FIG. 7B, the coating 110 is positioned over two sides of the surface 114 of a strut 112. The coating 110 is applied on all surfaces 114 of the strut 112 in FIG. 7B, although alternative embodiments provide for a coating 110 applied selectively to one or more surface 114 of the strut 112, or applied selectively to only a portion of a strut surface 114. In one embodiment, the coating 110 includes a bioactive material 118 positioned between the surface 114 and a biocompatible material 120. The biocompatible material 120 may be selected to provide a desired rate of release of the bioactive material 118 from the coating 110.

In one aspect, the biocompatible material 120 is a porous biostable material. The biocompatible material 120 may include one or more biostable materials selected from the group consisting of parylene or a parylene derivative, acrylate polymers, amides of (meth)acrylic acid, N-vinyl compounds, vinyl esters of aliphatic monocarboxylic acids, butanediol-1,4-divinyl ether, butanediol-1,4-divinyl allyl ether, butanediol-1,4-divinyl allyl ester, the reaction product of butanediol-1 with (meth)acrylic acid, the reaction product of 4-diglycidyl ether with (meth)acrylic acid, the reaction product of bisphenol A diglycidyl ether with (meth)acrylic acid, polyalkylene oxalates, polyphosphazenes, polymonoacrylates, polyurethanes, silicones, polyesters, polyolefins, ethylene-alpha-olefin copolymers, acrylic polymers, vinyl halide polymers, polyvinyl ethers, polyvinylidene halides, polyvinyl ketones, polyvinyl aromatics, polyvinyl esters, acrylate copolymers, methylene methacrylate copolymers, ethylene methyl methacrylate copolymers, acrylonitrile-styrene copolymers, ABS resins, ethylene-vinyl acetates, polyamides, alkyd resins, polycarbonates, polyoxymethylenes, polyimides, polyethers, epoxy resins, polyurethanes, cellulose ethers and polymers, copolymers or mixtures thereof. Specific examples of preferred biostable biocompatible materials 120 include: arylene, poly(n-butyl-acrylate),poly(n-butyl methacrylate), poly 2-ethylhexyl acrylate, poly lauryl-acrylate, poly 2-hydroxy-propyl acrylate, polyvinyl chloride, polyvinyl methyl ether, polyvinylidene fluoride, polyvinylidene chloride, polyacrylonitrile, polystyrene, polyvinyl acetate, ethylenemethyl methacrylate copolymers, acrylonitrile-styrene copolymers, ethylene glycol diacrylate, ethylene glycol dimethacrylate, trimethylopropane triacrylate, trimethylopropane trimethacrylate, pentaerythritol tetraacrylate or pentaerythritol tetramethacrylate, 1,6-hexanediol dimethacrylate, diethyleneglycol dimethacrylate, N-methylol methacrylamide butyl ether, N-vinyl pyrrolidone, vinyl oleate, polyvinyl chloride, polyvinylidene fluoride, ABS resins, Nylon 66, rayon, rayon-triacetate, cellulose, cellulose acetate, cellulose butyrate, cellulose acetate butyrate, cellophane, cellulose nitrate, cellulose propionate, carboxymethyl cellulose, and polymers, copolymers or mixtures thereof. Particularly preferred biostable biocompatible materials 120 include a material selected from the group consisting of: a copolymer comprising styrene and isobutylene, phosphatidylcholine, and poly(butylmethacrylate).

In another aspect, the biocompatible material 120 is a bioabsorbable material. The bioabsorbable material preferably comprises one or more materials selected from the group consisting of: polydioxanone, polyorthoester, polyanhydride, polyphosphoester, polyphosphoester urethane, poly(amino acids), cyanoacrylates, poly(trimethylene carbonate), poly(iminocarbonates), copoly(ether-esters), polyalkylene oxalates, polyphosphazenes, polyesters, polyamides and copolymers or mixtures thereof.

In yet another aspect, the biocompatible material 120 is a mixture of a biostable material and a bioabsorbable material, such that dissolution of the bioabsorbable material upon implantation of the coating in the body vessel results in the formation of pores through the biocompatible material 120. The bioactive material 118 may elute through the pores formed in the biocompatible material 120 upon dissolution of the bioabsorbable material within the biostable material matrix. Examples of suitable bioabsorbable biocompatible materials 120 include one or more polymers selected from the group consisting of: poly(L-lactic acid), polycaprolactone, poly(lactide-co-glycolide), poly(hydroxybutyrate), poly(hydroxybutyrate-co-valerate), poly(glycolic acid), poly(D,L-lactic acid), poly(glycolic acid-co-trimethylene carbonate), poly(trimethylene carbonate), poly(ethylene oxide),poly(D-lactic acid), fibrin, fibrinogen, cellulose, starch, collagen, hyaluronic acid and copolymers or mixtures thereof. Preferred bioabsorbable materials include: poly(L-lactic acid), poly(lactide-co-caprolactone) copolymer, poly(lactide-co-glycolide) copolymer.

The coating may further include materials positioned between the surface of the medical device and the bioactive material. FIG. 7C shows an second coating 110′ comprising the bioactive material 118 and biocompatible material 120 positioned over a surface 114 of a strut 112, as described with respect to FIG. 7B. However, the second coating 110′ further comprises a second biocompatible material 116 positioned between the bioactive material 118 and the surface 114 of the strut 112. The second biocompatible material 116 may be selected to adhere the bioactive material 118 to the surface 114, or to moderate the rate of release of the bioactive material 118 from the surface 114. Examples of suitable materials for the second biocompatible material 116 include parylene and silane.

The biostable material 120 may optionally include one or more bioactive matierials, and preferably has a thickness of between about 0.5 micrometers and about 25 micrometers thick. In alternative embodiments, the bioactive material 118 may optionally include one or more bioabsorbable or biostable biocompatible materials. In one embodiment, the coating comprises two or more layers. For example, a coating 110 may be formed by depositing a bioactive material 118 optionally mixed with a biostable polymer and/or a bioabsorbable polyer over at least a portion of the surface 114 of a medical device, or over a biocompatible material 116 that is deposited on at least a portion of the surface 114 of the medical device. Optionally, a biocompatible material 120 having biostable and/or bioabsorbable materials may be deposited over the bioactive material 118. In one example, the coating 110 includes two or three layers each comprising the bioactive material 118. Alternatively, the coating 110 may include a first layer positioned between a second layer and the surface 114 of the medical device. The first layer may comprise or consist essentially of the bioactive material 118, and may optionally include a biocompatible material selected to regulate the release of the bioactive material 118. The second layer may comprise or consist essentially of a biocompatible material 120 configured to regulate the release of the bioactive material 118 in the first layer. Optionally, the second layer may further include a bioactive material 118 that is the same or different from the bioactive material in the first layer. For example, in one aspect, the coating 110 comprises a layer comprising paclitaxel in the first layer and a layer comprising poly(lactic acid), or other suitable bioabsorbable material, in the second layer. The first layer may also include a mixture of paclitaxel and poly(lactic acid). Alternatively, the first layer may comprise a polybutylmethacrylate biostable polymer and a bioactive material such as rapamycin or a rapamycin derivative. In another aspect, the coating 110 is a three-layer coating, as illustrated in FIG. 7C such that the third layer of bioacompatible material 116 is preferably parylene; the bioactive material 118 includes both rapamycin (or a rapamycin analog) and a biostable polymer, and the second layer (biocompatible material 120) is formed from a porous biostable polymer.

Optionally, one or more portions of the device may include apertures or partially enclosed regions within the device for containing the bioactive material (such as wells, grooves or holes) into which a bioactive material is placed. Optionally, one or more biocompatible materials (116, 120) may also be included within the aperture. The one or more biocompatible materials (116, 120) may optionally be mixed with the bioactive material in the aperture. The apertures may be formed in the surface of the device by any suitable technique. For example, such techniques include drilling or laser cutting, electron-beam machining and the like or employing photoresist procedures and etching the desired apertures.

In a particularly preferred aspect, the layer of bioactive material contains from about 0.1 to 10.0 μg/mm², more preferably about 1.0 to 5.0 μg/mm², and in the present embodiment was about 3.0 μg/mm² of the gross surface area of the structure. “Gross surface area” refers to the area calculated from the gross or overall extent of the structure, and not necessarily to the actual surface area of the particular shape or individual parts of the structure. In other terms, about 100 μg to about 300 μg of drug per 0.001 inch of coating thickness may be contained on the device surface.

1. A collapsible vena cava filter for introduction into a blood vessel of a patient comprising:
an apical hub;
a plurality of struts secured to and diverging from said apical hub, each of said plurality of struts terminating in holding mechanisms that engage the walls of the blood vessel to secure the filter in a selected location therein;
a bioactive coating defining an exterior surface of said filter and configured to contact the walls of the blood vessel to prevent the growth of tissue that would interfere with removal of the filter; and
wherein said bioactive coating includes about 0.1 to 10.0 μg of bioactive material per 1.0 mm² of a coated surface area of the filter.

2. A collapsible vena cava filter for introduction into a blood vessel of a patient as set forth in claim 1 wherein the bioactive coating is paclitaxel.

3. A collapsible vena cava filter for introduction into a blood vessel of a patient as set forth in claim 1 wherein the bioactive coating is dexamethasone or related compounds.

4. A collapsible vena cava filter for introduction into a blood vessel of a patient as set forth in claim 1 wherein the exterior surface defined by the bioactive coating includes surfaces of the apical hub and struts that could engage the vessel wall.

5. A collapsible vena cava filter for introduction into a blood vessel of a patient as set forth in claim 1 wherein the exterior surface defined by the bioactive coating includes the entire exterior surface of the filter.

6. A collapsible vena cava filter for introduction into a blood vessel of a patient as set forth in claim 1 wherein an excipient may be associated with said bioactive coating.

7. A collapsible vena cava filter for introduction into a blood vessel of a patient as set forth in claim 4 wherein the bioactive coating is paclitaxel.

8. A collapsible vena cava filter for introduction into a blood vessel of a patient as set forth in claim 1 wherein the bioactive coating includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic materials; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic material; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C—, ³H—, ¹³¹I—, ³²P— or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar® or other agents for treating benign prostatic hyperplasia (BHP) or a mixture of any of these; and various forms of small intestine submucosa (SIS).

9. A collapsible vena cava filter for introduction into a blood vessel of a patient comprising:
an apical hub;
a plurality of struts secured to and diverging from said apical hub, each of said plurality of struts terminating in holding mechanisms that engage the walls of the blood vessel to secure the filter in a selected location therein;
a bioactive coating defining an exterior surface of said filter and configured to contact the walls of the blood vessel to prevent the growth of tissue that would interfere with removal of the filter; and
wherein said layer of bioactive coating contains about 100 μg to about 300 μg of drug per 0.001 inch of coating thickness.

10. A collapsible vena cava filter for introduction into a blood vessel of a patient as set forth in claim 9 wherein the bioactive coating is paclitaxel.

11. A collapsible vena cava filter for introduction into a blood vessel of a patient as set forth in claim 9 wherein the bioactive coating is dexamethasone or related compounds.

12. A collapsible vena cava filter for introduction into a blood vessel of a patient as set forth in claim 9 wherein the exterior surface defined by the bioactive coating includes surfaces of the apical hub and struts that could engage the vessel wall.

13. A collapsible vena cava filter for introduction into a blood vessel of a patient as set forth in claim 9 wherein the exterior surface defined by the bioactive coating includes the entire exterior surface area of the filter.

14. A collapsible vena cava filter for introduction into a blood vessel of a patient as set forth in claim 12 wherein the bioactive coating is paclitaxel.

15. A collapsible vena cava filter for introduction into a blood vessel of a patient as set forth in claim 9 wherein the bioactive coating includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic materials; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic material; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C—, ³H—, ¹³¹I—, ³²P— or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar® or other agents for treating benign prostatic hyperplasia (BHP) or a mixture of any of these; and various forms of small intestine submucosa (SIS).

16. A collapsible filter for introduction into a blood vessel of a patient, said collapsible filter having a proximal portion, a medial portion and a distal portion, comprising:
an apical hub, in the proximal portion of said filter, having a first or distal end and a second or proximal end;
a plurality of struts having proximal end and distal end portions, the proximal ends of said plurality of struts being secured to the first or distal end of said apical hub and diverging distally and outwardly therefrom, and each of said struts having an outwardly turned hook at their distal ends;
a pair of side elements associated with each of said struts, each side element having a proximal portion and a distal portion, the proximal end of the proximal portions being secured to the first or distal end of said apical hub and diverging distally and outwardly therefrom such that the associated strut lies between the pair of side elements, the distal portion of each side element diverging inwardly toward said associated strut such that the distal ends of the pair of side elements meet and form an eyelet through which the associated strut passes in a sliding relationship, whereby the filter as a whole may be unfolded from a collapsed insertion condition in which the struts and side elements form a narrow bundle for arrangement in a catheter like insertion instrument into an open tulip like filter configuration with the side elements interoposed between the struts;
a deployment and retrieval section secured to and extending proximately from the second or proximal end of said apical hub;
a bioactive coating defining an exterior surface of said filter to prevent the growth of tissue that would interfere with removal of the filter; and
wherein the bioactive coating includes about 0.1 to 10.0 μg of bioactive material per 1.0 mm² of a coated surface area of the filter.

17. A collapsible filter for introduction into a blood vessel as set forth in claim 16, wherein:
said bioactive coating defines the exterior surface of the distal end portion of the struts and their hooks to prevent the ingrowth of tissue to and therearound.

18. A collapsible filter for introduction into a blood vessel as set forth in claim 17, wherein:
said bioactive coating defines the exterior surface of said first or distal end of the apical hub and the deployment and retrieval section that is secured to the apical hub to prevent the ingrowth of tissue to and therearound.

19. A collapsible filter for introduction into a blood vessel as set forth in claim 16, wherein:
said bioactive coating defines the entire exterior surface area of the filter to prevent the ingrowth of tissue to and therearound.

20. A collapsible filter for introduction into a blood vessel as set forth in claim 16 wherein:
said bioactive coating is dexamethasone.

21. A collapsible filter for introduction into a blood vessel as set forth in claim 16 wherein:
said bioactive coating is pacilitaxel.

22. A collapsible filter for introduction into a blood vessel of a patient as set forth in claim 15 wherein the layer of bioactive coating contains about 100 μg to about 300 μg of drug per 0.001 inch of coating thickness.

23. A collapsible filter for introduction into a blood vessel of a patient as set forth in claim 16 wherein the layer of bioactive coating includes at least one of heparin, covalent heparin, or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; Hytrin® or other antihypertensive agents; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethyl sulfoxide (DMSO), a retinoid or another antisecretory agent; cytochalasin or another actin inhibitor; or a remodelling inhibitor; deoxyribonucleic acid, an antisense nucleotide or another agent for molecular genetic intervention; methotrexate or another antimetabolite or antiproliferative agent; tamoxifen citrate, Taxol® or the derivatives thereof, or other anti-cancer chemotherapeutic materials; dexamethasone, dexamethasone sodium phosphate, dexamethasone acetate or another dexamethasone derivative, or another anti-inflammatory steroid or non-steroidal antiinflammatory agent; cyclosporin or another immunosuppressive agent; trapidal (a PDGF antagonist), angiopeptin (a growth hormone antagonist), angiogenin, a growth factor or an anti-growth factor antibody, or another growth factor antagonist; dopamine, bromocriptine mesylate, pergolide mesylate or another dopamine agonist; ⁶⁰Co (5.3 year half life), ¹⁹²Ir (73.8 days), ³²P (14.3 days), ¹¹¹In (68 hours), ⁹⁰Y (64 hours), ^{99m}Tc (6 hours) or another radiotherapeutic material; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-aminosteroid (lasaroid) or another free radical scavenger, iron chelator or antioxidant; a ¹⁴C—, ³H—, ¹³¹I—, ³²P— or ³⁶S-radiolabelled form or other radiolabelled form of any of the foregoing; estrogen or another sex hormone; AZT or other anti polymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin or other antibody targeted therapy agents; gene therapy agents; and enalapril and other prodrugs; Proscar® or other agents for treating benign prostatic hyperplasia (BHP) or a mixture of any of these; and various forms of small intestine submucosa (SIS).

24. A collapsible vena cava filter for introduction into a blood vessel of a patient as set forth in claim 1 wherein said bioactive coating includes about 1.0 to 5.0 μg of bioactive material per 1.0 mm² of the coated surface area of the filter.

25. A collapsible vena cava filter for introduction into a blood vessel of a patient as set forth in claim 24 wherein said bioactive coating includes about 3.0 μg of bioactive material per 1.0 mm² of the coated surface area of the filter.

26. A collapsible filter for introduction into a blood vessel of a patient as set forth in claim 16 wherein said bioactive coating includes about 1.0 to 5.0 μg of bioactive material per 1.0 mm² of the coated surface area of the filter.

27. A collapsible filter for introduction into a blood vessel of a patient as set forth in claim 26 wherein said bioactive coating includes about 3.0 μg of bioactive material per 1.0 mm² of the coated surface area of the filter.

28. A collapsible filter for introduction into a blood vessel of a patient as set forth in claim 16 wherein each of the pair of side elements is a unitary, one-piece component.
